(19) United States
(12) Patent Application Publication
(10) Pub. No.: US 2005/0019317 A1
(43) Pub. Date: Jan. 27, 2005

(54) Composition for human immunostimulation containing Lysozyme and Lactal Bumin

(76) Inventor: Bridget Qiuying Cheng, West Covina, CA (US)

Correspondence Address: LAW OFFICES OF J.F. LEE, 17800 CASTLETON STREET, SUITE 383, CITY OF INDUSTRY, CA, 91748, US

(21) Appl. No.: 10/625,013

(22) Filed: Jul. 22, 2003

(51) Int. Cl. A61K 38/47

(52) U.S. Cl. 424/94.61

(57) ABSTRACT

A composition for human immunostimulation or immunomodulation containing the ingredients of Lysozyme and Lactal Bumin, made into capsules. The capsules can be taken orally 2 to 3 times a day.

### FIELD OF THE INVENTION

The present invention relates generally to a food supplement to be taken orally for increasing the strength of human immune system. Said food supplement composition contains the ingredients of Lysozyme and Lactal Bumin

### BACKGROUND OF THE INVENTION

It was known for quite a long period of time that Lysozyme, a material found in chicken egg white and human tears, has some capability in reducing the viral and bacterial viability. Lysozyme is a bacteriolytic protein that is found to help the dissolution of cell walls, especially those of microorganisms and bacteria.

Use of Lysozyme as medical treatment is known in the medical and pharmaceutical community, with additional components added into the composition. For example, Russian researchers once showed the utility of Lysozyme to control infections of the upper digestive tract when used as a chewing gum. This was reported in Callerio Foundation's website at www.callerio.org.

Lysozyme can also be found in the hemolymph of insects that are known to have strong resistance to bacterial infections. Lysozyme's lytic attribute, used as antimicrobial agent to fight bacteria, has further been applied to produce genetic transformation of plant species with better disease/pest resistance. Reference U.S. Pat. No. 5,597,945.

Coupled with other ingredients, such as Lactal Bumin in present invention, that have synergistic effect with Lysozyme, the compositions formed are found to be inductive of human body's defense mechanism; this is generally known as immunostimulation or immunomodulation.

Although compositions of Lysozyme and other materials, such as Bromelain (extracted from pineapple), have been attempted and made before, the composition of Lysozyme and Lactal Bumin, in the ratio specified in present invention was not known or attempted before.

### OBJECTS AND SUMMARY OF THE INVENTION

One object of present invention seeks to introduce a new composition of food supplement having therapeutic value in human immunostimulation or immunomodulation. Said composition is made up of 10% Lysozyme and 90% Lactal Bumin.

In particular, present invention is directed toward the production of a capsule weighing 200 mg, containing 20 mg of Lysozyme, and 180 mg of Lactal Bumin. Said capsule then is suggested for oral consumption 2 to 3 times a day, at even time intervals.

The oral administration of the capsule (200 mg) in present invention will vary according to the health status of the user. The capsule can be served, with water, to patents with common cold, tonsillitis, cancer, Hepatitis B, or other unidentifiable malaises.

For patients with moderate symptoms, the dosage is two capsules a day (morning/evening), 30 minutes after meals. For patients with severe symptoms, two capsules can be served in the morning and evening, respectively, after meal.

For cancer patients, two capsules are served three times a day after each meal.

### DETAILED DESCRIPTION OF THE PREFERRED EMBODIMENT

Step 1. Weight, check and record all the ingredients.
Step 2. Screen all lumpy materials through # 40-50 mesh screen.
Step 3. Charge a suitable blender, blend all components in the blender until uniform. Which means the mixture of two main ingredients will be 180 mg Lactal Bumin and 20 mg Lysozyme per (200 mg) capsule.
Step 4. Unload all blended powder and pack into suitable container lined with clean poly bags.
Step 5. Weigh, check and record the actual total yield. Find actual total yield of the product is checked against theoretical total yield limits. (Theoretical total yield limits is 97% 100.5% of the theoretical total yield)
Step 6. Encapsulate the above blended powder into # 2 clear/clear two pieces hardshell and pass through the polisher.
Step 7. Unload all polished capsules into 9±½″×9½″×14¼″ corrugated boxes lined with 20″×30″×1.5 mils clean poly bags.
Step 8. Follow all USP-NF specifications, pack capsules into bottles.

1. A composition for human immunostimulation comprising about 10% by weight of Lysozyme and 90% by weight of Lactal Bumin.

2. The composition of claim 1, wherein the specific weight makeup in a capsule of 200 mg is 20 mg of Lysozyme and 180 mg of Lactal Bumin.
